(19) United States
(12) Patent Application Publication
(10) Pub. No.: US 2011/0245587 A1
(43) Pub. Date: Oct. 6, 2011

(54) METHOD FOR CORRECTION OF FEMALE URINARY INCONTINENCE AND SKIN REDUCTION

(76) Inventor: Julie Ann Reil, Billings, MT (US)

(21) Appl. No.: 12/754,466

(22) Filed: Apr. 5, 2010

(51) Int. Cl. A61F 2/02

(52) U.S. Cl. 600/30

(57) ABSTRACT

A novel method of treating female incontinence by use of nonablative, broadband near-infrared light applied to seven defined treatment areas of the female external genitalia to achieve skin reduction and improve skin tone of the urethra and the supportive tissues surrounding the urethra, thereby treating urinary incontinence by improving both intrinsic and extrinsic urethral support non-surgically, non-invasively, without pain, without anesthesia and without recovery time. This method represents the first use of this technology as a medical treatment, outside of cosmetic use. This method solves the problems related or inherent to surgical treatment options for female incontinence because the advantage of this technology, with light penetration just 1 to 3 mm into the skin, is that there is no cutting or trauma to the skin and there is no potential for complications such as urethral scarring, over-correction, over-tightening or obstruction. This novel method is low risk and low cost to patients, low risk for doctors, and capable of significantly lowering the burden of suffering and cost from urinary incontinence having both treatment and preventative applications for urinary incontinence.

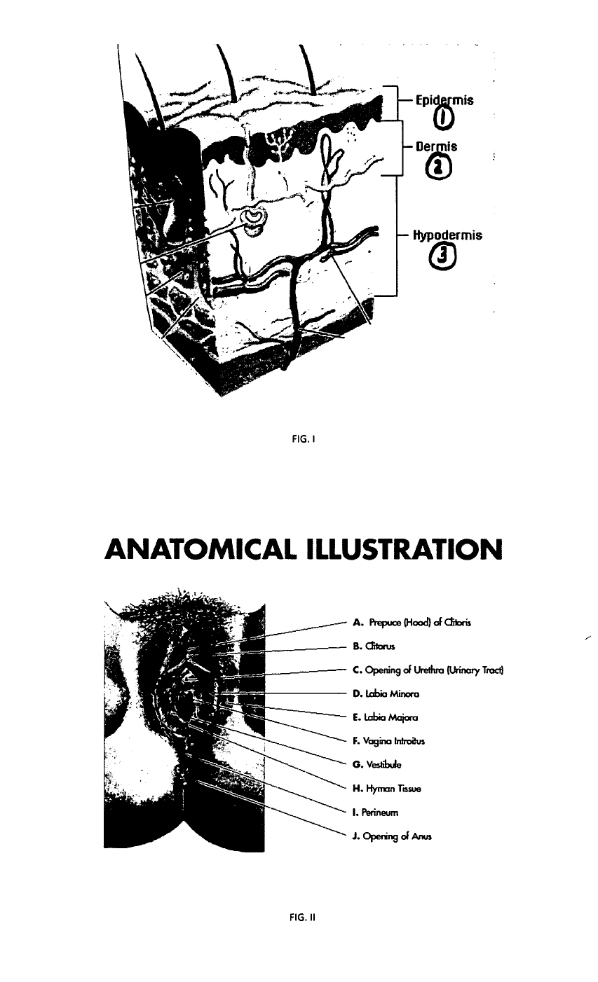

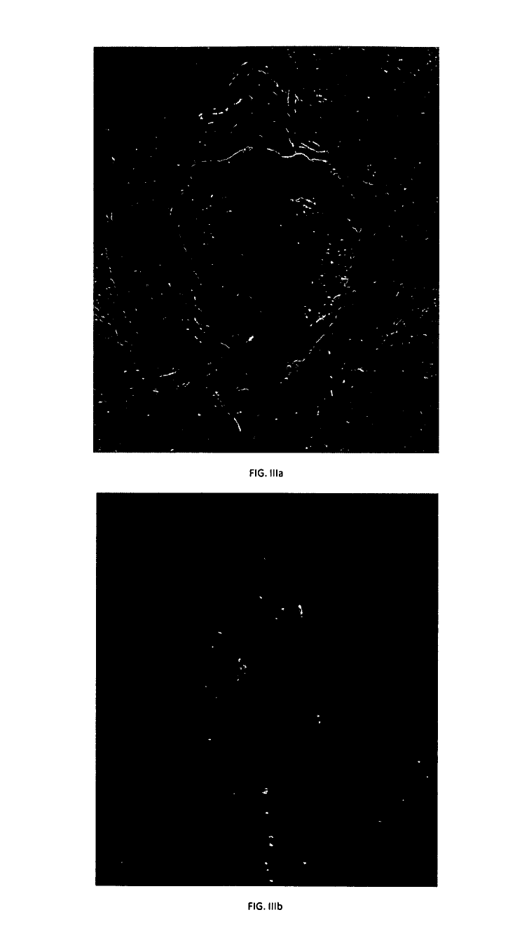

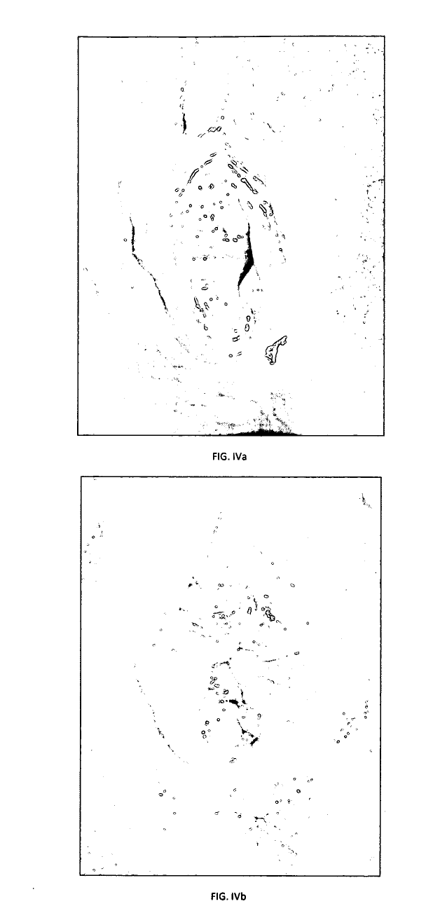

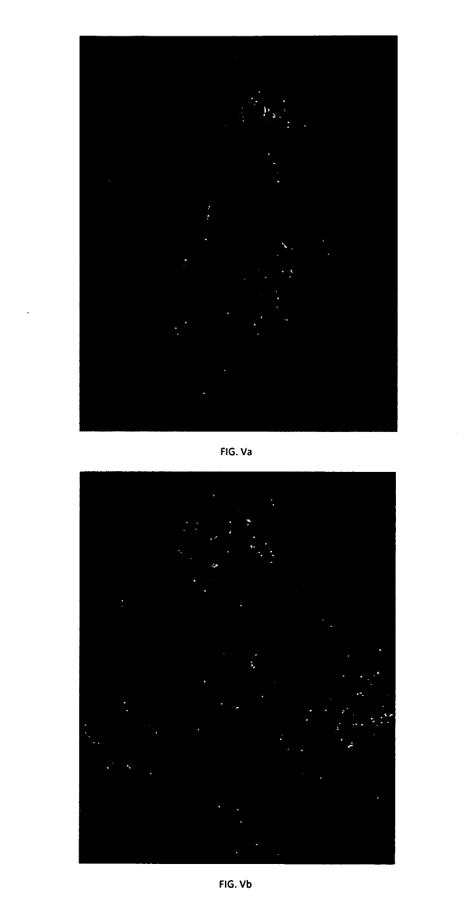

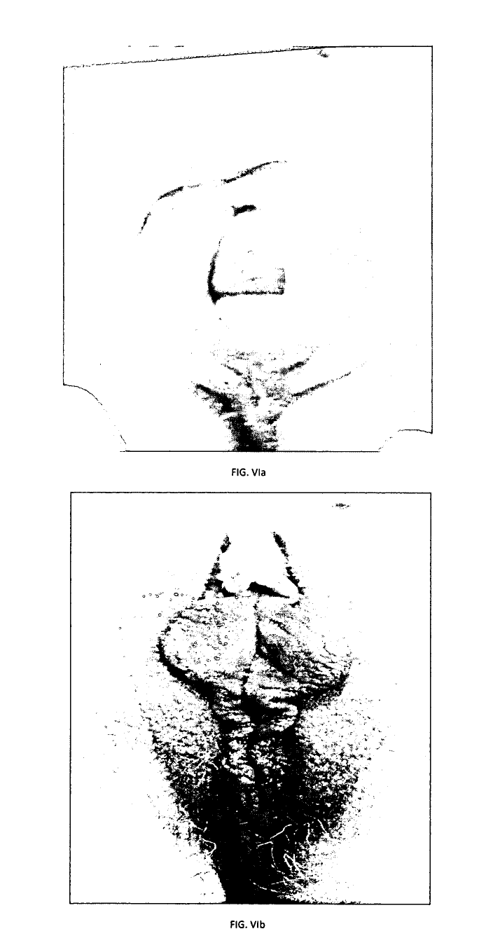

### BACKGROUND OF THE INVENTION

The present invention pertains to a novel method of female genital skin reduction and improvement of skin tone using near-infrared light for the treatment of female urinary incontinence.

This invention relates to a novel method for correction of female incontinence.

This novel Invention addresses a global problem afflicting an estimated 200 million people worldwide. (5) The problem is urinary incontinence. Urinary incontinence is a stigmatized, underreported, under-diagnosed, under-treated medical condition that is erroneously thought to be a normal part of aging and up to one in four women over the age of 18 experience episodes of leaking urine involuntarily. (5)

The type of incontinence that statistically affects most women, which is the focus of medical and surgical procedures for the correction of female incontinence, stress urinary incontinence, is the leaking of urine during physical movements such as coughing, sneezing, walking or exercising, when there is pressure on the bladder.

Childbirth, menopause and aging can weaken the pelvic floor muscles, the vagina and the ligaments that support the bladder. When the supporting structures are weakened, the bladder and vaginal walls can move downward, altering the urethral position and keeping the muscles from squeezing as tightly as they normally could. Without a tightly sealed urethra, urine can leak during movements of physical stress.

Another type of incontinence, urge incontinence, losing urine after inappropriate bladder spasms or contractions of the detrusor muscle, usually at unexpected times such as touching or hearing the sound of water, during sleep, or after drinking water, is commonly treated with anti-cholinergic medications.

Very common among post-menopausal women is mixed incontinence, a combination of stress and urge incontinence. Most women do not have pure stress or pure urge incontinence and it has been suggested that mixed incontinence may be the most common type of urinary incontinence among women.

Much less common is overactive bladder, when nerves send signals to the bladder at the wrong time, day and night, when the bladder is not full, causing the muscles to squeeze without warning.

Relatively rare in women are overflow incontinence, when the bladder doesn't empty properly causing urine to spill over, and functional incontinence, which happens to women with impaired thinking, moving or communicating making it hard for them to reach the toilet.

Transient incontinence is leaking that occurs temporarily due to a medication effect, urinary tract infection or restricted mobility from an injury. A respiratory infection can trigger transient incontinence, which resolves when coughing ends.

Both stress and urge incontinence become more frequent with age and are most prevalent at the time of menopause, approximately age 50, and again at age 65. (2) Obesity, multiple pregnancies/deliveries and white race are known to be risk factors for incontinence, with obese women having twice the risk of leaking compared to non-obese women. (1)

The social costs of urinary incontinence are high and even mild symptoms will affect social, sexual, interpersonal, and professional function. (5) Many women make adaptations to their activity level and even stop participating in exercise to avoid embarrassment, which impacts their health, overall fitness level and quality of life. Maintaining an active lifestyle is an important aspect to treating incontinence.

Incontinence can lead to feelings of shame and embarrassment and lead to low self esteem. Intimate relationships are often affected because of urine odor, pad use and frequent trips to the toilet. The fear of a major leaking accident when in public leads most incontinence sufferers to eventually become socially isolated. 53% of homebound older persons are incontinent and more than half of all residents in nursing homes are incontinent, with incontinence being the second leading cause of institutionalization in the U.S. and the cost of caring for urinary incontinence in nursing facilities estimated at $5.3 billion. (5)

The costs to our healthcare system and to society from incontinence are riveting. In 1995, the societal cost of incontinence for individuals over 65 years of age and older was $26.3 billion, or $3,565 per individual with urinary incontinence, with most of the total cost associated with direct treatment, such as diagnostic testing and medication. (5)

The medical and surgical solutions for the problem of incontinence are invasive, require either local or general anesthesia, hospitalization, require time for recovery and healing, involve significant potential risks including hemorrhage, prolonged urinary retention, infection, urethral obstruction, de novo urge incontinence, damage to the surrounding tissue and erosion through tissue. The medical and surgical solutions presently available to women treat severe, daily symptoms of urinary incontinence and but do not treat preventatively.

Medical and Surgical treatments for female urinary incontinence are customized methods aimed at improving either intrinsic urethral tone or improving extrinsic urethral tone.

Prior art such as U.S. Pat. No. 5,112,344 discloses a method for surgical treatment of female urinary incontinence where a looping filamentary element is placed between the vaginal wall and rectus abdominis sheath and passed on each side of the urethra in an attempt to correct the urethral position by encouraging the development of a scar tissue, thereby improving extrinsic urethral support.

Other prior art, U.S. Pat. No. 5,899,909, discusses a surgical method used to treat female urinary incontinence where tape is passed into the body through the vagina on either side of the urethra to form a loop around the urethra, which is tightened and attached to the abdominal wall, in an attempt to give the urethra added extrinsic support and tone.

Additional prior art, U.S. Pat. No. 6,406,423 discloses another method for surgical treatment for urinary incontinence. This method involves forming openings suprapubically and vaginally and forming tracks, verifying tracks by cystoscopy, passing a sleeved tape through these tracks to form a loop under the urethra, tightening the loop, removing the sleeve and leaving the tape implanted under the urethra, to give the urethra added extrinsic support and tone.

Other prior art, U.S. Pat. No. 7,112,171 discusses a sling assembly with secure and convenient attachment, as an improved and potentially safer instrument for performing the urethral sling surgical method, a procedure involving placement of a sling made of mesh or tape to stabilize or support the urethra extrinsically. The sling procedure has potential complications of urethral obstruction, development of de novo urge incontinence, hemorrhage, prolonged urinary retention, infection and damage to the surrounding tissue and sling erosion, as discussed in the above patent background.

There are other concerns regarding the sling device. Many midurethral slings and related devices have received approval from the FDA through a 510(k) process that does not require proof of safety and efficacy of the new device, but requires evidence that something similar has already been approved for use, and after a particular sling device was approved through a 510(k) process and put into use before clinical trials were conducted, this device unfortunately resulted in erosion through the vaginal wall, causing pain and bleeding for women, and had to be removed from the market. (1)

Prior art, U.S. Pat. No. 5,957,920 discusses in the background other options for treatment of urinary incontinence including injection of collagen around the urethra attempting to improve intrinsic tone of the urethra. This patent describes a novel technique or method for treating urinary incontinence using radiofrequency waves to thermally damage cells of the internal urethra, thereby promoting scar tissue, attempting to improve intrinsic urethral tone.

The referenced medical publication from the author of this patent, used the method introduced here in a medical investigation of thirty women, each having a series of three treatments, with most frequently reported results being a) longer bladder holding capacity, b) improved support in the female genital area and c) improved urinary control with physical exertion. (4)

This background information has been provided to give information believed by this applicant to be of possible relevance to this new, novel method presented here.

When the pelvic floor muscles, connective tissue and skin of the female genitals are weakened by an increase of pressure on the pelvic muscles during pregnancy, or by maximal stretching of the pelvic tissues during childbearing, or by loss of estrogen effect on genital tissues after menopause, or by general laxity due to aging, the intrinsic (functional) urethral tone and extrinsic (structural) urethral position are affected which lead to urinary incontinence.

Medical and Surgical treatments for female urinary incontinence have focused on methods for improving either intrinsic urethral tone or extrinsic urethral tone. The novel method introduced here improves both intrinsic urethral tone and extrinsic urethral tone, a dual treatment function not previously achieved with a single treatment.

This novel invention addresses both structural and functional deficits which cause urinary incontinence.

A functional urethra, one with optimal tone, forms an adequate seal to stop involuntary leaking of urine. When urethral support is compromised functionally, the tone of the urethra is not adequate to maintain a tight seal to stop urine from leaking under pressure, for instance when the bladder is full. This functional deficit, decreased intrinsic urethral tone, is corrected by this novel treatment method.

A structurally supported urethra is surrounded by pelvic floor muscles, connective tissue and skin structures that lend support for optimal positioning of the urethra. When urethral support is compromised structurally, the position of the urethra becomes mobile during movements, referred to as urethral hyper-mobility, and leaking occurs with coughing, sneezing, laughing and even walking. This structural deficit, decreased extrinsic urethral tone, is corrected by this novel treatment method.

This method solves the problems related or Inherent to surgical procedures because it is completely non-invasive: i) no injection of foreign substances, ii) no anesthesia required, iii) no urethral overcorrection or over tightening, iv) no urethral scarring or obstruction, v) no risk of infection or hemorrhage, vi) no foreign body use, vii) no cutting or trauma to the skin.

The current methods for treatment of female urinary incontinence are invasive, require recovery time and are expensive. These methods also involve potential risks including the injection or placement of foreign substances or objects into the body, general anesthesia, overcorrection of the urethral tone leading to urethral obstruction, development of new urge incontinence, infection, hemorrhage, as well as scarring and erosion of a foreign body through the urethral tissues resulting in chronic pain and bleeding.

The current methods of treatment of female urinary incontinence are not effective in preventing urinary incontinence, which is known to be a condition that progressively worsens as women advance in age through menopause and beyond. There is a need for a completely non-invasive treatment for urinary incontinence and for a treatment that can be used to treat women in the early stages of incontinence as well as treat incontinence preventatively.

The present invention provides a novel method which overcomes the deficiencies and risks of previous medical and surgical procedures for female urinary incontinence. This invention not only overcomes those shortcomings but also provides features and advantages not previously found in other methods and technologies.

The method for this invention is a new and novel medical procedure that is completely non-invasive, does not require hospitalization or extended recovery, in fact, there is no recovery time, and women of any age and any skin color with mild, moderate and severe symptoms of urinary incontinence are candidates for this medical procedure. The method for this procedure results in a wide variety of medical improvements within the focused treatment area.

The primary benefits and advantages of this new invention are: i) non-invasive, ii) requires no anesthesia, iii) requires no recovery time, iv) treats mild, moderate and severe incontinence

The novelty of this new invention is that all women regardless of their age, skin color or severity of symptoms of involuntary leaking of urine, mild, moderate and severe, are candidates for this medical procedure. With no hospitalization required and no recovery time required for this low-risk out-patient procedure, the cost savings are a tremendous advantage for the patient, for the doctor, and for the health care system with malpractice risk being minimal as a result of the excellent safety record of the technology used to perform the procedure. Healthcare, hospital care, medical costs to employers and employees are on the increase which means that the average woman cannot afford the cost of a surgical procedure or the time away from work during recovery.

This novel medical procedure eliminates the high costs and morbidity associated with the present surgical treatment options available for stress urinary incontinence.

This novel invention can provide an effective and safe medical treatment for female urinary incontinence, and can also be used for prevention of female incontinence, which has far-reaching implications for improvement of physical fitness, health, well-being, and quality of life of women and cost savings to the health care system in preventing the cost burden related the progressively worsening medical condition of incontinence.

### BRIEF SUMMARY OF THE INVENTION

It will become apparent that this invention introduces a new, non-invasive medical procedure using methods and technology applied to the female genitalia that have never before been used for the treatment of urinary incontinence in a female patient.

The technology used for this treatment method, widely used in cosmetic medicine, Pub. No. US 2005/0049658 and Pub. No. US 2006/0052847, uses a broadband near-infrared light source and is capable of controlling skin temperature, which therefore eliminates the risk of thermal injury to the patient by protecting the epidermis during treatment.

To the best of this author's knowledge, this method represents the first non-cosmetic use or medical treatment using this technology.

The technology uses a near-infrared light source with a spectrum of 1100 to 1800 nanometers where the light is absorbed by water in the skin at a depth of 1 to 3 millimeters. Through sustained heating of the dermis for up to 6 seconds, there is immediate collagen contraction and long term stimulation of collagen remodeling, thus reducing skin, tightening skin and improving skin tone.

The biggest advantage to this technology which uses light penetrating only 1-3 mm in skin depth, is that treatments are not painful and require no anesthesia, which is distinctly different from radiofrequency treatments, which can penetrate beyond 20 mm's in skin depth and are described as painful by patients.

Before this nonablative, infrared device was available, nonablative radiofrequency was the only method for skin tightening. In one study, this infrared device not only effectively caused skin contraction at lower fluencies, but also worked below the threshold of pain, which is a tremendous advantage of this technology. (3)

The advantages of this treatment method when compared to other medical and surgical treatments currently available: i) completely non-invasive, ii) non-painful, iii) no recovery time, iv) able to treat women of all skin color and ages, v) able to treat women with any severity of incontinence symptoms including mild, moderate and severe, vi) low risk, out-patient medical procedure, vii) time saving and cost saving for patients, doctors and for the health care system, viii) able to be used as a preventative for urinary incontinence after childbirth, ix) results in additional benefits to the treatment area including improved pelvic muscle contraction, xi) improvement of skin resistance to breakdown from urine, xii) this method results in additional benefits to women overall with improved self esteem and improved active lifestyle for women afflicted with urinary incontinence.

The objective of this invention is to introduce a new medical procedure, that is completely non-invasive, painless and safe, for treatment of urinary incontinence, for use by doctors worldwide, that is low cost to patients, low risk to patients, low risk medical malpractice and capable of significantly eliminating the burden of suffering and cost from urinary incontinence with both treatment and preventative applications.

### BRIEF DESCRIPTION OF THE SEVERAL VIEWS OF THE DRAWING

Benefits and advantages of the novel method of the present invention will be apparent from the following description accompanying drawings, FIG. I and FIG. II. Due to the limitation and inability to clearly illustrate the effect of skin reduction and improvement of skin tone with this method using an ink drawing, black and white scanned photographs; FIG. IIIa, FIG. IIIb, FIG. IVa, FIG. IVb, FIG. Va, FIG. Vb, FIG. VIa, FIG. VIb, have been submitted to demonstrate the invention more clearly.

FIG. I is a schematic illustration of the skin structure.

FIG. II is a schematic illustration of the female genitalia.

FIG. IIIa is female genitalia before treatment.

FIG. IIIb is female genitalia after treatment.

FIG. IVa is female genitalia before treatment.

FIG. IVb is female genitalia after treatment.

FIG. Va is female genitalia before treatment.

FIG. Vb is female genitalia after treatment.

FIG. VIa is female genitalia before treatment.

FIG. VIb is female genitalia after treatment.

### DETAILED DESCRIPTION OF THE INVENTION

The description of this new, novel method is as follows:

Infrared light is used to heat the dermal layer of the skin (see FIG. I-2). Sustained heating of the dermal layer of the skin over several seconds contracts the collagen and elastin components of the dermis resulting in tightening of skin, reduction of skin, and improved skin tone. (see FIG. IIIa, FIG. IIIb, FIG. IVa, FIG. IVb, FIG. Va, FIG. Vb)

The procedure is performed with application of the light device to seven treatment area (FIG. II-A, FIG. II-C, FIG. II-D, FIG. II-E, FIG. II-F, FIG. II-I, FIG. II-J). The description of the procedure application to each of these seven treatment areas is as follows:

Application of the light device to the treatment area labeled in FIG. II-E is as follows. The light device is applied to the treatment area just after a layer of cooled ultrasound gel is generously applied to the skin being treated. When the treatment window through which the infrared light is emitted is placed firmly onto the skin, the pulse is started. Within the span of several seconds the skin is pre-cooled, heated with infrared light and post-cooled. This is referred to as a ‘pulse’. Next, pulses are continued with minimal overlapping until the entire treatment area has been covered. The series of pulses required to completely cover a treatment area is referred to as a “pass”. Upon completion of a pass, the cool gel is wiped away, replaced with a new layer of cooled ultrasound gel over the treatment area, and the area is again covered in pulses to complete a pass. This process continues until the optimal number of passes are completed, which completes treatment of area FIG. II-E. Treatment of area FIG. II-E results in some immediate reduction in skin and ongoing reduction of skin over several weeks time. The procedure can be used advantageously to preferentially reduce and tone skin more on one side of the treatment area that on the other side to improve labia majora symmetry for women who have significant size discrepancy between the two sides of the labia majora.

The application of the light device to the treatment area labeled in FIG. II-D. The light device is applied to the treatment area just after a layer of cooled ultrasound gel is generously applied to the skin being treated. When the treatment window through which the infrared light is emitted is placed firmly onto the skin, the pulse is started. Within the span of several seconds the skin is pre-cooled, heated with infrared light and post-cooled. This is referred to as a ‘pulse’. Next, pulses are continued with minimal overlapping until the entire treatment area has been covered. The series of pulses required to completely cover a treatment area is referred to as a “pass”. Upon completion of a pass, the cool gel is wiped away, replaced with a new layer of cooled ultrasound gel over the treatment area, and the area is again covered in pulses to complete a pass. This process continues until the optimal number of passes are completed, which completes treatment of area FIG. II-D. Treatment of area FIG. II-D results in some immediate reduction in skin and ongoing reduction of skin over several weeks time. The procedure can be used advantageously to preferentially reduce skin more on one side of the treatment area that on the other to improve labia minora symmetry for women who have significant size discrepancy between the two sides of the labia minora.

The application of the light device to the treatment area labeled in FIG. II-A. The light device is applied to the treatment area just after a layer of cooled ultrasound gel is generously applied to the skin being treated. When the treatment window through which the infrared light is emitted is placed firmly onto the skin, the pulse is started. Within the span of several seconds the skin is pre-cooled, heated with infrared light and post-cooled. This is referred to as a ‘pulse’. Next, pulses are continued with minimal overlapping until the entire treatment area has been covered. The series of pulses required to completely cover a treatment area is referred to as a “pass”. Upon completion of a pass, the cool gel is wiped away, replaced with a new layer of cooled ultrasound gel over the treatment area, and the area is again covered in pulses to complete a pass. This process continues until the optimal number of passes are completed, which completes treatment of area FIG. II-A. Treatment of area FIG. II-A results in some immediate reduction in skin and ongoing reduction of skin over several weeks time.

The application of the light device to the treatment area labeled in FIG. II-C, periurethral and urethral skin. The light device is applied to the treatment area just after a layer of cooled ultrasound gel is generously applied to the skin being treated. When the treatment window through which the infrared light is emitted is placed firmly onto the skin, the pulse is started. Within the span of several seconds the skin is pre-cooled, heated with infrared light and post-cooled. This is referred to as a ‘pulse’. Next, pulses are continued with minimal overlapping until the entire treatment area has been covered. The series of pulses required to completely cover a treatment area is referred to as a “pass”. Upon completion of a pass, the cool gel is wiped away, replaced with a new layer of cooled ultrasound gel over the treatment area, and the area is again covered in pulses to complete a pass. This process continues until the optimal number of passes are completed, which completes treatment of area FIG. II-C. Treatment of area FIG. II-C results in some immediate reduction in skin and ongoing reduction of skin over several weeks time.

The application of the light device to the treatment area labeled in FIG. II-F. The light device is applied to the treatment area just after a layer of cooled ultrasound gel is generously applied to the skin being treated. When the treatment window through which the infrared light is emitted is placed firmly onto the skin, the pulse is started. Within the span of several seconds the skin is pre-cooled, heated with infrared light and post-cooled. This is referred to as a ‘pulse’. Next, pulses are continued with minimal overlapping until the entire treatment area has been covered. The series of pulses required to completely cover a treatment area is referred to as a “pass”. Upon completion of a pass, the cool gel is wiped away, replaced with a new layer of cooled ultrasound gel over the treatment area, and the area is again covered in pulses to complete a pass. This process continues until the optimal number of passes are completed, which completes treatment of area FIG. II-F. Treatment of area FIG. II-F results in some immediate reduction in skin and ongoing reduction of skin over several weeks time. The procedure can be used advantageously to preferentially reduce skin of the vaginal introitus and distal vaginal sidewalls for improvement of cystocele and rectocele. (See FIG. VIa, FIG. VIb)

The application of the light device to the treatment area labeled in FIG. II-I. The light device is applied to the treatment area just after a layer of cooled ultrasound gel is generously applied to the skin being treated. When the treatment window through which the infrared light is emitted is placed firmly onto the skin, the pulse is started. Within the span of several seconds the skin is pre-cooled, heated with infrared light and post-cooled. This is referred to as a ‘pulse’. Next, pulses are continued with minimal overlapping until the entire treatment area has been covered. The series of pulses required to completely cover a treatment area is referred to as a “pass”. Upon completion of a pass, the cool gel is wiped away, replaced with a new layer of cooled ultrasound gel over the treatment area, and the area is again covered in pulses to complete a pass. This process continues until the optimal number of passes are completed, which completes treatment of area FIG. II-I. Treatment of area FIG. II-I results in some immediate reduction in skin and ongoing reduction of skin over several weeks time. The procedure can be used advantageously to improve perinea! scarring.

The application of the light device to the treatment area labeled in FIG. II-1. The light device is applied to the treatment area just after a layer of cooled ultrasound gel is generously applied to the skin being treated. When the treatment window through which the infrared light is emitted is placed firmly onto the skin, the pulse is started. Within the span of several seconds the skin is pre-cooled, heated with infrared light and post-cooled. This is referred to as a ‘pulse’. Next, pulses are continued with minimal overlapping until the entire treatment area has been covered. The series of pulses required to completely cover a treatment area is referred to as a “pass”. Upon completion of a pass, the cool gel is wiped away, replaced with a new layer of cooled ultrasound gel over the treatment area, and the area is again covered in pulses to complete a pass. This process continues until the optimal number of passes are completed, which completes treatment of area FIG. II-1. Treatment of area FIG. II-1 results in some immediate reduction in skin and ongoing reduction of skin over several weeks time. The procedure can be used advantageously to improve external hemorrhoids and anal tone.

Therefore the scope of the following invention is limited only by the following claims. The embodiments of the invention being thus described it will be obvious that the same may be varied in many ways. Such variations are not to be regarded as a departure from the spirit and scope of the invention, and all such modifications as would be obvious to one skilled in the art are intended to be included within the scope of the following claims.

1. A method of treating urinary incontinence comprising:
a. cool gel application to specific treatment area followed by
b. dermal heat application to specific treatment area resulting in reduction of skin, which tightens skin and improves skin tone.

2. The method of 1 applied to the labia majora treatment area E results in reduction of skin.

3. The method of 1 applied to the labia minora treatment area D results in reduction of skin.

4. The method of 1 applied to the clitoral hood treatment area A results in reduction of skin.

5. The method of 1 applied to the periurethral and urethral skin treatment area C results in reduction of skin.

6. The method of 1 applied to the vaginal introitus treatment area F results in reduction of skin.

7. The method of 1 applied to the perineal treatment area I results in reduction of skin.

8. The method of 1 applied to the anal treatment area J results in reduction of skin.

9. The method of 1 further comprising:
a. a treatment pulse which is the combination of pre-surface cooling, dermal heating created by a sustained pulse of light and post-surface cooling
b. a treatment pass which is the summation of treatment pulses used to cover a treatment area.

10. The method of claim 9 results in improved urinary holding capacity.

11. The method of claim 9 results in improved urinary control.

12. The method of claim 9 results in improved urethral tone.

13. The method of claim 9 results in improved urethral position.

14. The method of claim 9 results in improvement of involuntary leaking of urine.

15. The method of claim 9 results in improvement of stress urinary incontinence.

16. The method of claim 9 results in improvement of urge urinary incontinence.

17. The method of claim 9 results in improvement of mixed urinary incontinence.

18. The method of claim 9 results in improvement of overflow incontinence.

19. The method of claim 9 results in improvement of perinea! scars.

20. The method of claim 9 results in improvement of external hemorrhoids.

21. The method of claim 9 results in improvement of rectal holding capacity.

22. The method of claim 9 results in improvement of anterior vaginal wall tone and improvement of cystocele.

23. The method of claim 9 results in improvement of posterior vaginal wall tone and improvement of rectocele.

24. The method of claim 9 results in improvement of vulvar varicosities.

25. The method of claim 9 results in improvement of pelvic muscle tone.

26. The method of claim 9 results in improvement of skin tone in all treatment areas.

27. The method of claim 9 results in improvement of skin condition and health in all treatment areas.
